SEQUENCE LISTING (15 sequences)

SEQ ID NO: 1 - protein, 122 aa, Artificial Sequence
  Synthetic construct
QVQLVQSGPEVKKPGASVKVSCKASGYTFTENSMHWVRQSHGKSLEWMGGINPNNGGTSYNQKFKGKVTMTTDKSTSTAYMELRSLTSDDTAVYYCARPYYYGYREDYFDYWGQGTTLTVSS

SEQ ID NO: 2 - protein, 107 aa, Artificial Sequence
  Synthetic construct
EIVMTQSPGTLSLSPGERATLSCRASSSVSYMYWYQQKPGQAPRPLIYLTSNLASGIPDRFSGSGSGTDYTLTISRLEPEDFAVYYCQQWSSYPPTFGQGTKVEIKR

SEQ ID NO: 3 - protein, 5 aa, Artificial Sequence
  Synthetic construct
ENSMH

SEQ ID NO: 4 - protein, 5 aa, Artificial Sequence
  Synthetic construct
EYTMH

SEQ ID NO: 5 - protein, 17 aa, Artificial Sequence
  Synthetic construct
GINPNNGGTSYNQKFKG

SEQ ID NO: 6 - protein, 17 aa, Artificial Sequence
  Synthetic construct
GIDPNNGGTWYNQKFKG

SEQ ID NO: 7 - protein, 17 aa, Artificial Sequence
  Synthetic construct
GINPNNGGTWYNQKFKG

SEQ ID NO: 8 - protein, 17 aa, Artificial Sequence
  Synthetic construct
GINPNNGGTWYNRKFKG

SEQ ID NO: 9 - protein, 13 aa, Artificial Sequence
  Synthetic construct
PYYYGYREDYFDY

SEQ ID NO: 10 - protein, 13 aa, Artificial Sequence
  Synthetic construct
PYYYGSREDYFDY

SEQ ID NO: 11 - protein, 10 aa, Artificial Sequence
  Synthetic construct
SASSSVSYMY

SEQ ID NO: 12 - protein, 10 aa, Artificial Sequence
  Synthetic construct
RASSSVSYMY

SEQ ID NO: 13 - protein, 8 aa, Artificial Sequence
  Synthetic construct
YLTSNLAS

SEQ ID NO: 14 - protein, 9 aa, Artificial Sequence
  Synthetic construct
QQWSSYPPT

SEQ ID NO: 15 - protein, 9 aa, Artificial Sequence
  Synthetic construct
QQWSSNPPT

(19) United States
(12) Patent Application Publication
(10) Pub. No.: US 2021/0017284 A1
(43) Pub. Date: Jan. 21, 2021

(54) HIGH AFFINITY NEUTRALIZING MONOCLONAL ANTIBODIES TO PROGRAMMED DEATH LIGAND 1 (PD-L1) AND USES THEREOF

(71) Applicant: ABEOME CORPORATION, Bogart, GA (US)

(72) Inventors: Richard SHIMKETS, Commerce, GA (US); Thomas VINCENT, Athens, GA (US); Crystal JACKSON, Jefferson, GA (US)

(21) Appl. No.: 16/982,295

(22) PCT Filed: Mar. 19, 2019
(86) PCT No.: PCT/US2019/022971

§ 371 Date: Sep. 18, 2020

Related U.S. Application Data
(60) Provisional application No. 62/644,832, filed on Mar. 19, 2018.

(51) Int. Cl. C07K 16/28

(52) U.S. Cl. CPC C07K 16/2827; C07K 2317/76; C07K 2317/565

(57) ABSTRACT

Disclosed herein are novel PD-L1 binding molecules and methods of their use. The disclosed PD-L1 binding molecules include neutralizing anti-PD-L1 antibodies, bi-specific antibodies, and immunotoxins. The description provides for the use of the disclosed PD-L1 binding molecules in the treatment, prevention, inhibition, or reduction of a cancer or metastasis or as part of a treatment regimen including the use of other anti-cancer agents.

This application claims the benefit of U.S. Provisional Application No. 62/644,832, filed on Mar. 19, 2018, which is incorporated herein by reference in its entirety.

### I. BACKGROUND

1. The currently available therapeutic strategy for the treatment of solid tumors is due, in large part, to the difficulty in achieving therapeutically effective levels of chemotherapeutic agents in the area of tumor growth and infiltration. Commonly, treatment is based on surgery and/or radiation therapy and/or chemotherapy, but these methods give unsatisfactory results in a significant percentage of cases. Thus, it is clear that there is a need for improving the currently available therapies for the treatment of cancers involving solid tumors and/or for developing new therapies targeting said solid tumors.

### II. SUMMARY OF THE INVENTION

2. Disclosed herein, in one aspect, are binding molecules directed to PD-L1 that suitable for use in the treatment of PD-L1 mediated diseases and disorders.

3. In one aspect, disclosed herein are isolated PD-L1 binding molecules comprising a heavy chain variable domain comprising one or more Complementary Determining Regions (CDR)s as set forth in TABLE 1 (for example, SEQ ID NO: 3, SEQ ID NO: 4, SEQ ID NO: 5, SEQ ID NO: 6, SEQ ID NO: 7, SEQ ID NO: 8, SEQ ID NO: 9, and/or SEQ ID NO: 10). For example, disclosed herein are isolated PD-L1 binding molecules (including, but not limited to neutralizing PD-L1 binding molecules such as, for example, neutralizing anti-PD-L1 antibodies) of any preceding aspect, wherein the binding molecule comprising a heavy chain variable domain comprises the CDRs as set forth in SEQ ID NO: 3 and SEQ ID NO: 5; SEQ ID NO: 3 and SEQ ID NO: 6; SEQ ID NO: 3 and SEQ ID NO: 7; SEQ ID NO: 3 and SEQ ID NO: 8; SEQ ID NO: 3 and SEQ ID NO: 9; SEQ ID NO: 3 and SEQ ID NO: 10; SEQ ID NO: 4 and SEQ ID NO: 5; SEQ ID NO: 4 and SEQ ID NO: 6; SEQ ID NO: 4 and SEQ ID NO: 7; SEQ ID NO: 4 and SEQ ID NO: 8; SEQ ID NO: 4 and SEQ ID NO: 9; SEQ ID NO: 4 and SEQ ID NO: 10; SEQ ID NO: 3, SEQ ID NO: 5, and SEQ ID NO: 9; SEQ ID NO: 3, SEQ ID NO: 6, and SEQ ID NO: 9; SEQ ID NO: 3, SEQ ID NO: 7, and SEQ ID NO: 9; SEQ ID NO: 3, SEQ ID NO: 8, and SEQ ID NO: 9; SEQ ID NO: 3, SEQ ID NO: 5, and SEQ ID NO: 10; SEQ ID NO: 3, SEQ ID NO: 6, and SEQ ID NO: 10; SEQ ID NO: 3, SEQ ID NO: 7, and SEQ ID NO: 10; SEQ ID NO: 3, SEQ ID NO: 8, and SEQ ID NO: 10; SEQ ID NO: 4, SEQ ID NO: 5, and SEQ ID NO: 9; SEQ ID NO: 4, SEQ ID NO: 6, and SEQ ID NO: 9; SEQ ID NO: 4, SEQ ID NO: 7, and SEQ ID NO: 9; SEQ ID NO: 4, SEQ ID NO: 8, and SEQ ID NO: 9; SEQ ID NO: 4, SEQ ID NO: 5, and SEQ ID NO: 10; SEQ ID NO: 4, SEQ ID NO: 6, and SEQ ID NO: 10; SEQ ID NO: 4, SEQ ID NO: 7, and SEQ ID NO: 10; or SEQ ID NO: 4, SEQ ID NO: 8, and SEQ ID NO: 10.

4. Also disclosed herein are isolated PD-L1 binding molecules of any preceding aspect further comprising light chain variable domains comprising one or more CDRs as set forth in TABLE 1 (for example, SEQ ID NO: 11, SEQ ID NO: 12, SEQ ID NO: 13, SEQ ID NO: 14, and/or SEQ ID NO: 15). For example, disclosed herein are isolated PD-L1 binding molecules (including, but not limited to neutralizing PD-L1 binding molecules such as, for example, neutralizing anti-PD-L1 antibodies) of any preceding aspect, wherein the binding molecule comprising a light chain variable domain comprises the CDRs as set forth in SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15.

5. In one aspect disclosed herein are PD-L1 binding molecules of any preceding aspect, wherein the PD-L1 molecule comprises both a heavy chain variable domain and a light chain variable domain or complementary determining regions (CDRs) of any preceding aspect, such as, for example a PD-L1 binding molecule comprising a heavy chain variable region as set forth in SEQ ID NO: 1 and/or a light chain variable region as set forth in SEQ ID NO: 2. In one aspect, the PD-L1 binding molecule of any preceding aspect can be an antibody (or fragment thereof) or an immunotoxin. In one aspect, disclosed herein are antibodies (or fragments thereof) of any preceding aspect, wherein the antibody (or fragment thereof) can be a neutralizing antibody.

6. Also disclosed herein are antibodies and/or immunotoxins comprising a heavy chain variable region as set forth in SEQ ID NO: 1 and/or a light chain variable region as set forth in SEQ ID NO: 2. In one aspect, disclosed herein are humanized versions of any antibody or immunotoxin of any preceding aspect.

7. Also disclosed herein are methods for treating, preventing, inhibiting, or reducing a cancer or metastasis in a subject, preferably a PD-L1-positive cancer or tumor, comprising the step of administering to said patient a therapeutically effective amount of an anti-PD-L1 antibody (including, but not limited to neutralizing PD-L1 binding molecules such as, for example, neutralizing anti-PD-L1 antibodies) and/or anti-PD-L1 immunotoxin of any preceding aspect.

### III. DETAILED DESCRIPTION

8. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

9. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

10. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

11. The term “subject” is defined herein to include animals such as mammals, including, but not limited to, primates (e.g., humans), cows, sheep, goats, horses, dogs, cats, rabbits, rats, mice and the like. In some embodiments, the subject is a human.

12. “Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

13. “Biocompatible” generally refers to a material and any metabolites or degradation products thereof that are generally non-toxic to the recipient and do not cause significant adverse effects to the subject.

14. “Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

15. A “control” is an alternative subject or sample used in an experiment for comparison purposes. A control can be “positive” or “negative.”

16. “Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

17. A “decrease” can refer to any change that results in a smaller gene expression, protein expression, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

18. “Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

19. The terms “prevent,” “preventing,” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

20. “Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

21. “Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

22. “Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

23. “Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

24. “Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

25. In this specification and in the claims which follow, reference will be made to a number of terms which shall be defined to have the following meanings:

26. “Optional” or “optionally” means that the subsequently described event or circumstance may or may not occur, and that the description includes instances where said event or circumstance occurs and instances where it does not.

27. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

28. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular anti-PD-L1 antibody is disclosed and discussed and a number of modifications that can be made to a number of molecules including the anti-PD-L1 antibody are discussed, specifically contemplated is each and every combination and permutation of an anti-PD-L1 antibody and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

29. T cells play an essential role in the anti-cancer immune response. T cell activation depends on the initial antigen-specific signal, presented via the antigen-loaded major histocompatibility complex (MHC) to the T cell receptor, and on activation of the costimulatory molecule CD28 by binding of CD80/86. T cells also express coinhibitory molecules that are capable of downregulating the immune response. One major coinhibitory receptor is programmed death 1 (PD-1).

30. The terms “PD1”, “PD-1” and “Programmed cell death protein 1” refer to a member of the CD28 superfamily that delivers negative signals upon interaction with its two ligands, PD-L1 or PD-L2. PD-1 and its ligands are broadly expressed and exert a wider range of immunoregulatory roles in T cells activation and tolerance compared with other CD28 members. PD-1 was isolated as a gene up-regulated in a T cell hybridoma undergoing apoptosis and was named program death 1. PD-1 has two ligands, programmed death ligand-1 (PD-L1) and PD-L2, of which PD-L1 is most widely expressed. PD-L1, also known as CD274, Programmed Cell Death 1 Ligand 1 (PDCD1LG1 or PD-L1) or B7-H1, is a type I transmembrane glycoprotein composed of IgC- and IgV-type extracellular domains, which binds to PD1.

31. Binding of PD-L1 to PD-1 transduces an inhibitory signal to the T cell, resulting in inhibition of T cell proliferation, reduced secretion of effector cytokines, and potentially exhaustion. By up-regulating PD-L1 expression levels, tumor cells are capable of escaping immune recognition and attack. PD-L1 is expressed on a wide variety of tumors, including breast cancer, gastric cancer, renal cell cancer, ovarian cancer, non-small lung cancer, melanoma, and hematological cancers. In general, PD-1 and PD-L1 have been demonstrated to be poor prognostic factors as high expression levels are associated with poor outcome of cancer patients. Preclinical studies with anti-PD-1 and anti-PD-L1 antibodies have shown promising anti-tumor effects and have led to the initiation of several clinical investigations. Early clinical trials demonstrated objective and durable (>1 year) responses in patients with treatment-refractory, advanced melanoma, renal cell carcinoma, non-small cell lung cancer, and ovarian cancer. Because of these impressive results, phase II/III studies are currently further exploring the therapeutic efficacy of these agents. Due to the impressive efficacy in melanoma patients, the FDA has recently granted accelerated approval of pembrolizumab (anti-PD-1 antibody) for the treatment of patients with advanced or unresectable melanoma following progression on prior therapies. Nevertheless, only about 30% of patients see successful outcomes with the presently available anti-PD-1 antibodies. Thus, new more effective and more universal anti-PD-1/PDL-1 antibodies, bi-specific antibodies and immunotoxins are needed.

32. Thus, the present disclosure relates to an anti-PD-L1 antibodies and immunotoxins for use in a method of treating a tumor, preferably a PD-L1-positive tumor, in a patient. Herein is shown that labelled anti-PD-L1 antibodies are targeted specifically to tumors expressing PD-L1. Anti-PD-L1 antibodies coupled to a toxin can therefore be used in therapy in order to target and kill tumor cells.

33. As used herein, the term ‘immunotoxin” has its general meaning in the art. By “immunotoxin”, it is meant a chimeric protein made of an antibody or modified antibody or antibody fragment (also called in the present application “antibody”), attached to a fragment of a toxin. The antibody of the immunotoxin is covalently attached to the fragment of a toxin. Preferably, the fragment of the toxin is linked by a linker to the antibody or fragment thereof. Said linker is preferably chosen from 4-mercaptovaleric acid and 6-maleimidocaproic acid.

34. The term “anti-PD-L1 immunotoxin” refers to an antibody-drug conjugate (ADC) wherein the antibody moiety is an anti-PD-L1 antibody and wherein said anti-PD-L1 antibody is linked to a toxin. Such a toxin could be a native or engineered toxin, and may have been de-immunized to reduce immunogenicity. Upon binding to PD-L1 on its target cells, the immunotoxin enters the cells and kills the target cells. As used herein, the term “antibody” has its general meaning in the art. The term “anti-PD-L1 antibody” refers to an antibody that binds specifically to PD-L1. Preferably, said antibody does not bind to PD-L2.

35. in one apect, the disclosed anti-PD-L1 antibodies could be fused to other binding molecules to create bi-specfic antibody molecules. Such molecules could utilize the PD-L1 binding component to target to PD-L1 positive tumors, and could be linked to antibodies to LAG-3, TGF-β, OX40, ICOS, LIGHT, and a variety of other T cell surface antigens.

Binding Molecules

36. As used herein the term “binding molecule” refers to an intact immunoglobulin including monoclonal antibodies, polyclonal antibodies, chimeric antibodies, humanized or human antibodies, as well as antibodies fragments and functional variants including antigen-binding and/or variable domain comprising fragment of an immunoglobulin that competes with the intact immunoglobulin for specific binding to the binding partner of the immunoglobulin, e.g. PD-L1.

37. In one aspect, the disclosed PD-L1 binding molecules can comprise an anti-PD-L1 antibody (for example, an anti-PD-L1 antibody). The term “antibodies” is used herein in a broad sense and includes both polyclonal and monoclonal antibodies. As used herein, the term “antibody” encompasses, but is not limited to, whole immunoglobulin (i.e., an intact antibody) of any class. In addition to intact immunoglobulin molecules, also included in the term “antibodies” are fragments or polymers of those immunoglobulin molecules, and human or humanized versions of immunoglobulin molecules or fragments thereof. Antibodies that bind the disclosed regions of PD-L1 involved in the interaction between PD-L1 and PD-1 are also disclosed.

38. It is understood and herein contemplated that the disclosed PD-L1 binding molecules (including binding molecules that comprise antibodies or a fragment thereof) bind PD-L1. However, it is further recognized that some PD-L1 binding molecules (including binding molecules that comprise antibodies or a fragment thereof) not only bind PD-L1, but also neutralize the biological effects of PD-L1 (i.e., they are neutralizing binding molecules). In one aspect, disclosed herein are PD-L1 binding molecules wherein the PD-L1 binding molecules comprise an anti-PD-L1 antibody that is a neutralizing antibody.

39. Native antibodies are usually heterotetrameric glycoproteins, composed of two identical light (L) chains and two identical heavy (H) chains. The disclosed PD-L1 binding molecules whether monoclonal antibodies, polyclonal antibodies, chimeric antibodies, humanized or human antibodies, as well as antibodies fragments and functional variants can comprise all or a portion of light and heavy chains.

40. In a complete antibody, typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant (C(H)) domains. Each light chain has a variable domain at one end (V(L)) and a constant(C(L)) domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (K) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

41. The term “variable” is used herein to describe certain domains of the heavy and light chains that differ in sequence among antibodies and are used in the binding and specificity of each particular antibody for its particular antigen. However, the variability is not usually evenly distributed through the variable domains of antibodies. The more highly conserved portions of the variable domains are called the framework (FR). The variable domains of native heavy and light chains each comprise four FR regions, largely adopting a n-sheet configuration, connected by three complementarity determining regions (CDRs), which form loops connecting, and in some cases forming part of, the n-sheet structure. The variability is typically concentrated in the CDRs or hypervariable regions both in the light chain and the heavy chain variable domains.

42. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The term “complementary determining regions” as used herein means sequences within the variable regions of binding molecules, such as immunoglobulins, that generate the antigen binding site which is complementary in shape and charge distribution to the epitope recognized on the antigen. The CDR regions can be specific for linear epitopes, discontinuous epitopes, or conformational epitopes of proteins or protein fragments, either as present on the protein in its native conformation or, in some cases, as present on the proteins as denatured, e.g., by solubilization in SDS. Epitopes may also consist of posttranslational modifications of proteins.

43. Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

44. CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically.

45. The constant domains are not involved directly in binding an antibody to an antigen, but exhibit various effector functions, such as participation of the antibody in antibody-dependent cellular toxicity.

In one aspect, the disclosed PD-L1 binding molecules (including, but not limited to neutralizing PD-L1 binding molecules such as, for example, neutralizing anti-PD-L1 antibodies) comprise one or more of the variable domain CDRs as shown in Table 1.

TABLE 1
PD-L1 binding molecules provided by their identification number,
heavy chain CDR1 sequence, heavy chain CDR2 sequence, heavy chain
CDR3 sequence, light chain CDR1 sequence, light chain CDR2 sequence,
and light chain CDR3 sequence.

| Name | CDR1 | CDR2 | CDR3 | CDR1 | CDR2 | CDR3 |
|---|---|---|---|---|---|---|
| ABM101 | ENSMH | GINPNNGGTSYNQK | PYYYGYREDYFDY | SASSSVSYMY | YLTSNLAS | QQWSSYPPT |
| | (SEQ ID | FKG | (SEQ ID NO: 9) | (SEQ ID NO: 11) | (SEQ ID NO: 13) | (SEQ ID NO: 14) |
| | NO: 3) | (SEQ ID NO: 5) | | | | |
| ABM102 | EYTMH | GIDPNNGGTWYNQK | PYYYGSREDYFDY | SASSSVSYMY | YLTSNLAS | QQWSSNPPT |
| | (SEQ ID | FKG | (SEQ ID NO: 10) | | | (SEQ ID NO: 15) |
| | NO: 4) | (SEQ ID NO: 9) | | | | |
| ABM103 | EYTMH | GINPNNGGTWYNQK | PYYYGSREDYFDY | SASSSVSYMY | YLTSNLAS | QQWSSNPPT |
| | | FKG | | | | |
| | | (SEQ ID NO: 7) | | | | |
| ABM118 | EYTMH | GINPNNGGTWYNQK | PYYYGSREDYFDY | SASSSVSYMY | YLTSNLAS | QQWSSNPPT |
| | | FKG | | | | |
| ABM139 | EYTMH | GINPNNGGTWYNRK | PYYYGSREDYFDY | SASSSVSYMY | YLTSNLAS | QQWSSNPPT |
| | | FKG | | | | |
| | | (SEQ ID NO: 8) | | | | |
| ABM101.1 | ENSMH | GINPNNGGTSYNQK | PYYYGYREDYFDY | RASSSVSYMY | YLTSNLAS | QQWSSYPPT |
| | | FKG | | (SEQ ID NO: 12) | | |

46. In one aspect, disclosed herein are isolated PD-L1 binding molecules (including, but not limited to neutralizing PD-L1 binding molecules such as, for example, neutralizing anti-PD-L1 antibodies) comprising a heavy chain variable domain comprising one or more Complementary Determining Regions (CDR)s as set forth in Table 1. Thus, for example, the one or more heavy chain variable domain CDRs can comprise SEQ ID NO: 3, SEQ ID NO: 4, SEQ ID NO: 5, SEQ ID NO: 6, SEQ ID NO: 7, SEQ ID NO: 8, SEQ ID NO: 9, and/or SEQ ID NO: 10. In one aspect, it is understood and herein contemplated that the PD-L1 binding molecules (including, but not limited to neutralizing PD-L1 binding molecules such as, for example, neutralizing anti-PD-L1 antibodies) disclosed herein can comprise heavy chain variable domain CDR comprising any combination of 2 or 3 heavy chain variable domain CDRs from the list of CDRs in Table 1. Thus, for example, the PD-L1 binding molecules (including, but not limited to neutralizing PD-L1 binding molecules such as, for example, neutralizing anti-PD-L1 antibodies) can comprise the heavy chain variable domain CDRs as set forth in SEQ ID NO: 3 and SEQ ID NO: 5; SEQ ID NO: 3 and SEQ ID NO: 6; SEQ ID NO: 3 and SEQ ID NO: 7; SEQ ID NO: 3 and SEQ ID NO: 8; SEQ ID NO: 3 and SEQ ID NO: 9; SEQ ID NO: 3 and SEQ ID NO: 10; SEQ ID NO: 4 and SEQ ID NO: 5; SEQ ID NO: 4 and SEQ ID NO: 6; SEQ ID NO: 4 and SEQ ID NO: 7; SEQ ID NO: 4 and SEQ ID NO: 8; SEQ ID NO: 4 and SEQ ID NO: 9; SEQ ID NO: 4 and SEQ ID NO: 10; SEQ ID NO: 3, SEQ ID NO: 5, and SEQ ID NO: 9; SEQ ID NO: 3, SEQ ID NO: 6, and SEQ ID NO: 9; SEQ ID NO: 3, SEQ ID NO: 7, and SEQ ID NO: 9; SEQ ID NO: 3, SEQ ID NO: 8, and SEQ ID NO: 9; SEQ ID NO: 3, SEQ ID NO: 5, and SEQ ID NO: 10; SEQ ID NO: 3, SEQ ID NO: 6, and SEQ ID NO: 10; SEQ ID NO: 3, SEQ ID NO: 7, and SEQ ID NO: 10; SEQ ID NO: 3, SEQ ID NO: 8, and SEQ ID NO: 10; SEQ ID NO: 4, SEQ ID NO: 5, and SEQ ID NO: 9; SEQ ID NO: 4, SEQ ID NO: 6, and SEQ ID NO: 9; SEQ ID NO: 4, SEQ ID NO: 7, and SEQ ID NO: 9; SEQ ID NO: 4, SEQ ID NO: 8, and SEQ ID NO: 9; SEQ ID NO: 4, SEQ ID NO: 5, and SEQ ID NO: 10; SEQ ID NO: 4, SEQ ID NO: 6, and SEQ ID NO: 10; SEQ ID NO: 4, SEQ ID NO: 7, and SEQ ID NO: 10; or SEQ ID NO: 4, SEQ ID NO: 8, and SEQ ID NO: 10. In one aspect, the PD-L1 binding molecule can comprise the variable heavy domain as set forth in SEQ ID NO: 1.

47. It is understood and herein contemplated that the disclosed complimentary determining regions of the heavy chain variable domains in the disclosed PD-L1 binding molecules can be contiguous or separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids. Thus, disclosed herein are PD-L1 binding molecules comprising heavy chain variable domains comprising at least two CDRs wherein the first CDR is separated from the second CDR by 10, 11, 12, 13, 14, 15, 16, 17, 18, 19, or 20 amino acids. and wherein the second CDR and the third CDR are separated by 25, 26, 27, 28, 29, 30, 31, 32, 33, 34, 35, 36, 37, 38, 39, or 40 amino acids.

48. It is understood and herein contemplated that the PD-L1 binding molecules (including, but not limited to neutralizing PD-L1 binding molecules such as, for example, neutralizing anti-PD-L1 antibodies) can comprise a light chain variable domain instead of or in addition to a heavy chain variable domain. In one aspect, disclosed herein are isolated PD-L1 binding molecules (including, but not limited to neutralizing PD-L1 binding molecules such as, for example, neutralizing anti-PD-L1 antibodies) comprising a light chain variable domain comprising one or more Complementary Determining Regions (CDR)s as set forth in Table 1. Thus, for example, the one or more light chain variable domain CDRs can comprise SEQ ID NO: 11, SEQ ID NO: 12, SEQ ID NO: 13, SEQ ID NO: 14, and/or SEQ ID NO: 15. In one aspect, it is understood and herein contemplated that the PD-L1 binding molecules (including, but not limited to neutralizing PD-L1 binding molecules such as, for example, neutralizing anti-PD-L1 antibodies) disclosed herein can comprise light chain variable domain CDR comprising any combination of 2 or 3 light chain variable domain CDRs from the list of CDRs in Table 1. Thus, for example, the PD-L1 binding molecules can comprise the light chain variable domain CDRs as set forth in SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15. In one aspect, the PD-L1 binding molecule can comprise the variable light chain domain as set forth in SEQ ID NO: 2.

49. It is understood and herein contemplated that the disclosed complimentary determining regions of the light chain variable domains in the disclosed PD-L1 binding molecules can be contiguous or separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids. Thus, disclosed herein are PD-L1 binding molecules comprising light chain variable domains comprising at least two CDRs wherein the first CDR is separated from the second CDR by 10, 11, 12, 13, 14, 15, 16, 17, 18, 19, or 20 amino acids. and wherein the second CDR and the third CDR are separated by 25, 26, 27, 28, 29, 30, 31, 32, 33, 34, 35, 36, 37, 38, 39, or 40 amino acids.

50. In one aspect, it is understood and herein contemplated that the PD-L1 binding molecules (including, but not limited to neutralizing PD-L1 binding molecules such as, for example, neutralizing anti-PD-L1 antibodies) disclosed herein can comprise a heavy chain variable domain and a light chain variable domain each domain comprising one or more Complementary Determining Regions (CDR)s as set forth in Table 1. Thus, for example, the PD-L1 binding molecule (including, but not limited to neutralizing PD-L1 binding molecules such as, for example, neutralizing anti-PD-L1 antibodies) can comprise any combination of one or more heavy chain variable domain CDRs as set forth SEQ ID NO: 3, SEQ ID NO: 4, SEQ ID NO: 5, SEQ ID NO: 6, SEQ ID NO: 7, SEQ ID NO: 8, SEQ ID NO: 9, and/or SEQ ID NO: 10 and one or more light chain variable domain CDRs as set forth in SEQ ID NO: 11, SEQ ID NO: 12, SEQ ID NO: 13, SEQ ID NO: 14 and/or SEQ ID NO: 15. Thus, for example, the PD-L1 binding molecules (including, but not limited to neutralizing PD-L1 binding molecules such as, for example, neutralizing anti-PD-L1 antibodies) can comprise the heavy chain variable domain CDR as set forth in SEQ ID NO: 3 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDR as set forth in SEQ ID NO: 4 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDR as set forth in SEQ ID NO: 5 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDR as set forth in SEQ ID NO: 6 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDR as set forth in SEQ ID NO: 7 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDR as set forth in SEQ ID NO: 8 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDR as set forth in SEQ ID NO: 9 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDR as set forth in SEQ ID NO: 10 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3 and SEQ ID NO: 5 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3 and SEQ ID NO: 6 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3 and SEQ ID NO: 7 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3 and SEQ ID NO: 8 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3 and SEQ ID NO: 9 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3 and SEQ ID NO: 10 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 4 and SEQ ID NO: 5 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 4 and SEQ ID NO: 6 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 4 and SEQ ID NO: 7 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 4 and SEQ ID NO: 8 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 4 and SEQ ID NO: 9 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 4 and SEQ ID NO: 10 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3, SEQ ID NO: 5, and SEQ ID NO: 9 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3, SEQ ID NO: 6, and SEQ ID NO: 9 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3, SEQ ID NO: 7, and SEQ ID NO: 9 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3, SEQ ID NO: 8, and SEQ ID NO: 9 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3, SEQ ID NO: 5, and SEQ ID NO: 10 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3, SEQ ID NO: 6, and SEQ ID NO: 10 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3, SEQ ID NO: 7, and SEQ ID NO: 10 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 3, SEQ ID NO: 8, and SEQ ID NO: 10 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 4, SEQ ID NO: 5, and SEQ ID NO: 9 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 4, SEQ ID NO: 6, and SEQ ID NO: 9 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 4, SEQ ID NO: 7, and SEQ ID NO: 9 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 4, SEQ ID NO: 8, and SEQ ID NO: 9 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 4, SEQ ID NO: 5, and SEQ ID NO: 10 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 4, SEQ ID NO: 6, and SEQ ID NO: 10 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; the heavy chain variable domain CDRs as set forth in SEQ ID NO: 4, SEQ ID NO: 7, and SEQ ID NO: 10 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15; or the heavy chain variable domain CDRs as set forth in SEQ ID NO: 4, SEQ ID NO: 8, and SEQ ID NO: 10 and the light chain variable domain CDRs as set forth in SEQ ID NO: 11; SEQ ID NO: 12; SEQ ID NO: 13; SEQ ID NO: 14; SEQ ID NO: 15; SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15. In one aspect, the PD-L1 binding molecule (including, but not limited to neutralizing PD-L1 binding molecules such as, for example, neutralizing anti-PD-L1 antibodies) can comprise the variable heavy chain domain as set forth in SEQ ID NO: 1 and the variable light chain domain as set forth in SEQ ID NO: 2.

51. As noted above the disclosed PD-L1 binding molecules (including, but not limited to neutralizing PD-L1 binding molecules such as, for example, neutralizing anti-PD-L1 antibodies) can also be fragments of antibodies. As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, dAb, complementarity determining region (CDR) fragments, single-chain antibodies (scFv), bivalent single-chain antibodies, diabodies or other bi-specific antibodies, triabodies, tetrabodies, (poly)peptides that contain at least a fragment of an immunoglobulin that is sufficient to confer specific antigen binding to the (poly)peptide, etc., including hybrid fragments. Thus, fragments of the antibodies that retain the ability to bind their specific antigens are provided. For example, fragments of antibodies which maintain PD-L1 binding activity are included within the meaning of the term “antibody or fragment thereof” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

52. Also included within the meaning of “antibody or fragments thereof” are conjugates of antibody fragments and antigen binding proteins (single chain antibodies). Conjugated antibodies or fragments refer to antibodies or fragments that are operatively linked or otherwise physically or functionally associated with an effector moiety or tag, such as inter alia a toxic substance, a radioactive substance, fluorescent substance, a liposome, or an enzyme as described, for example, in U.S. Pat. No. 4,704,692, the contents of which are hereby incorporated by reference.

53. Regardless of structure, the antigen-binding fragments disclosed herein can bind with the same antigen that is recognized by the intact immunoglobulin. An antigen-binding fragment can comprise a peptide or polypeptide comprising an amino acid sequence of at least 2 contiguous amino acid residues, at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 35 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least 200 contiguous amino acid residues, or at least contiguous 250 amino acid residues of the amino acid sequence of the binding molecule.

54. The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

55. The term “functional variant”, as used herein, refers to a binding molecule that comprises a nucleotide and/or amino acid sequence that is altered by one or more nucleotides and/or amino acids compared to the nucleotide and/or amino acid sequences of the parent binding molecule and that is still capable of competing for binding to the binding partner, e.g. PD-L1 (including PD-L1), with the parent binding molecule. In other words, the modifications in the amino acid and/or nucleotide sequence of the parent binding molecule do not significantly affect or alter the binding characteristics of the binding molecule encoded by the nucleotide sequence or containing the amino acid sequence, i.e. the binding molecule is still able to recognize and bind its target. The functional variant may have conservative sequence modifications including nucleotide and amino acid substitutions, additions and deletions. These modifications can be introduced by standard techniques known in the art, such as site-directed mutagenesis and random PCR-mediated mutagenesis, and may comprise natural as well as non-natural nucleotides and amino acids.

56. As disclosed herein, the binding molecules, antibodies, fragments, and variants are able to specifically bind to an antigenic target, such as, for example, PD-L1. The term “specifically binding”, as used herein, in reference to the interaction of a binding molecule, e.g. an antibody, and its binding partner, e.g. an antigen, means that the interaction is dependent upon the presence of a particular structure, e.g. an antigenic determinant or epitope, on the binding partner. In other words, the antibody preferentially binds or recognizes the binding partner even when the binding partner is present in a mixture of other molecules. The binding may be mediated by covalent or non-covalent interactions or a combination of both. In yet other words, the term “specifically binding” means immunospecifically binding to an antigen or a fragment thereof and not immunospecifically binding to other antigens. A binding molecule that immunospecifically binds to an antigen may bind to other peptides or polypeptides with lower affinity as determined by, e.g., radioimmunoassays (RIA), enzyme-linked immunosorbent assays (ELISA), BIAcore, or other assays known in the art. Binding molecules or fragments thereof that immunospecifically bind to an antigen may be cross-reactive with related antigens. Preferably, binding molecules or fragments thereof that immunospecifically bind to an antigen do not cross-react with other antigens.

57. In one aspect, the disclosed antibodies or binding molecules disclosed herein can be human antibodies or human binding molecules. The term “human”, when applied to binding molecules as defined herein, refers to molecules that are either directly derived from a human or based upon a human sequence. When a binding molecule is derived from or based on a human sequence and subsequently modified, it is still to be considered human as used throughout the specification. In other words, the term human, when applied to binding molecules is intended to include binding molecules having variable and constant regions derived from human germline immunoglobulin sequences based on variable or constant regions either or not occurring in a human or human lymphocyte or in modified form. Thus, the human binding molecules may include amino acid residues not encoded by human germline immunoglobulin sequences, comprise substitutions and/or deletions (e.g., mutations introduced by for instance random or site-specific mutagenesis in vitro or by somatic mutation in vivo). “Based on” as used herein refers to the situation that a nucleic acid sequence may be exactly copied from a template, or with minor mutations, such as by error-prone PCR methods, or synthetically made matching the template exactly or with minor modifications. Semisynthetic molecules based on human sequences are also considered to be human as used herein.

58. Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

59. Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

60. The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

61. In some aspect, it can be important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

62. Disclosed are hybridoma cells that produces the monoclonal antibody. The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

63. Monoclonal antibodies may be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises PD-L1. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of PD-L1 expressed as a fusion protein with human IgG1 is injected into the host animal according to methods known in the art (e.g., Kilpatrick K E, et al. Gene gun delivered DNA-based immunizations mediate rapid production of murine monoclonal antibodies to the Flt-3 receptor. Hybridoma. 1998 December; 17(6):569-76; Kilpatrick K E et al. High-affinity monoclonal antibodies to PED/PEA-15 generated using 5 microg of DNA. Hybridoma. 2000 August; 19(4):297-302, which are incorporated herein by referenced in full for the the methods of antibody production) and as described in the examples.

64. An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing domains of an anti-PD-L1 antibody as fusion proteins. The antigen is produced by inserting a gene fragment in-frame between the signal sequence and the mature protein domain of the anti-PD-L1 antibody nucleotide sequence. This results in the display of the foreign proteins on the surface of the virion. This method allows immunization with whole virus, eliminating the need for purification of target antigens.

65. Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against PD-L1. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane Antibodies, A Laboratory Manual Cold Spring Harbor Publications, New York, (1988).

66. After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

67. The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

68. The term “isolated”, when applied to binding molecules as defined herein, refers to binding molecules that are substantially free of other proteins or polypeptides, particularly free of other binding molecules having different antigenic specificities, and are also substantially free of other cellular or tissue material and/or chemical precursors or other chemicals. For example, when the binding molecules are recombinantly produced, they are preferably substantially free of culture medium, and when the binding molecules are produced by chemical synthesis, they are preferably substantially free of chemical precursors or other chemicals, i.e., they are separated from chemical precursors or other chemicals which are involved in the synthesis of the protein. Preferably, substantially free means that the binding molecule will typically comprise about 50%, 60%, 70%, 80% or 90% W/W of a sample, more usually about 95%, and preferably will be over 99% pure.

69. The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Optionally, such a non-immunoglobulin polypeptide is substituted for the constant domains of an antibody or substituted for the variable domains of one antigen-combining site of an antibody to create a chimeric bivalent antibody comprising one antigen-combining site having specificity for PD-L1 (including PD-L1) and another antigen-combining site having specificity for a different antigen.

70. In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

71. The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

72. Alternatively, the disclosed antibodies can be made utilizing transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

73. An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

74. One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

75. For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

76. Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

77. Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with PD-L1. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

78. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

79. A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

80. Also provided is an antibody reagent kit comprising containers of the monoclonal antibody or fragment thereof and one or more reagents for detecting binding of the anti-PD-L1 antibody or fragment thereof to the PD-L1 molecule. The reagents can include, for example, fluorescent tags, enzymatic tags, or other tags. The reagents can also include secondary or tertiary antibodies or reagents for enzymatic reactions, wherein the enzymatic reactions produce a product that can be visualized.

1. Homology/Identity

81. It is understood that one way to define any known variants and derivatives or those that might arise, of the disclosed genes and proteins herein is through defining the variants and derivatives in terms of homology to specific known sequences. For example, Table 4 sets forth a particular sequence of an PD-L1 heavy chain variable domain. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

82. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

83. It is understood that any of the methods typically can be used and that in certain instances the results of these various methods may differ, but the skilled artisan understands if identity is found with at least one of these methods, the sequences would be said to have the stated identity, and be disclosed herein.

84. For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

2. Peptides

a) Protein Variants

85. As discussed herein there are numerous variants of the PD-L1 binding molecules and PD-L1 binding CDRs and heavy and light chain variable regions disclosed herein that are known and herein contemplated. In addition, to the known functional strain variants there are derivatives of the PD-L1 binding molecules and PD-L1 binding CDRs and heavy and light chain variable regions which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. As used herein, “insertions” refer to a change in an amino acid or nucleotide sequence resulting in the addition of one or more amino acid or nucleotide residues, respectively, as compared to the parent, often the naturally occurring, molecule. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Table 5 and are referred to as conservative substitutions.

TABLE 4
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 5
Amino Acid Substitutions Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

86. Substantial changes in function or immunological identity are made by selecting substitutions that are less conservative than those in Table 5, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. Conservative amino acid substitutions include the ones in which the amino acid residue is replaced with an amino acid residue having similar structural or chemical properties. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

87. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

88. The replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

89. Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

90. Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T.E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

91. It is understood that one way to define the variants and derivatives of the disclosed proteins herein is through defining the variants and derivatives in terms of homology/identity to specific known sequences. For example, SEQ ID NOs: 1 and 2. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

92. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

93. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

94. It is understood that the description of conservative mutations and homology can be combined together in any combination, such as embodiments that have at least 70% homology to a particular sequence wherein the variants are conservative mutations.

95. As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. In addition, for example, a disclosed conservative derivative of SEQ ID NOs:1, 2, 3, 4,5,6,7,8,9,10, 11, 12, 13, or 14 such as the substitution of an isoleucine (I) at for a valine (V). It is understood that for this mutation all of the nucleic acid sequences that encode this particular derivative of the SEQ ID NOs:1, 2, 3, 4,5,6,7,8,9,10, 11, 12, 13, or 14, are also disclosed.

96. It is understood that there are numerous amino acid and peptide analogs which can be incorporated into the disclosed compositions. For example, there are numerous D amino acids or amino acids which have a different functional substituent then the amino acids shown in Table 4 and Table 5. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

97. Molecules can be produced that resemble peptides, but which are not connected via a natural peptide linkage. For example, linkages for amino acids or amino acid analogs can include CH₂NH—, —CH₂S—, —CH₂—CH₂—CH═CH— (cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. I 307-314 (1982) (—CH—CH—, cis and trans); Almquist et al. J Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

98. Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

99. D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

100. In one aspect, the disclosed PD-L1 binding molecules may further comprise a label. As used herein, a label can include a fluorescent dye, a member of a binding pair, such as biotin/streptavidin, a metal (e.g., gold), radioactive substituent, or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts.

101. Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type’ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin EBG; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

102. A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

103. The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

104. Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

105. As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

106. Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

3. Pharmaceutical Carriers/Delivery of Pharmaceutical Products

107. As described above, the compositions can also be administered in vivo in a pharmaceutically acceptable carrier (also referred to herein as a pharmaceutically acceptable excipient). By “pharmaceutically acceptable” is meant a material that is not biologically or otherwise inert, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. Thus, in one aspect, disclosed herein are pharmaceutical compositions comprising any of the PD-L1 binding molecules disclosed herein.

108. The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

109. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

110. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

111. The compositions, including antibodies, can be used therapeutically in combination with a pharmaceutically acceptable carrier.

112. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

113. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

114. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

115. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

116. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

117. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

118. Compositions for oral administration include powders or granules, suspensions or solutions in water or non-aqueous media, capsules, sachets, or tablets. Thickeners, flavorings, diluents, emulsifiers, dispersing aids or binders may be desirable.

119. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

4. Therapeutic Uses and Methods of Treatment

120. Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above. 121. In one aspect, it is understood and herein contemplated that any of the herein disclosed PD-L1 binding molecules (including, but not limited to neutralizing PD-L1 binding molecules such as, for example, neutralizing anti-PD-L1 antibodies) can be used to treat, prevent, inhibit, or reduce any disease where uncontrolled cellular proliferation occurs such as cancers and metastasis. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. 122. In one aspect, disclosed herein are methods of treating, preventing, inhibiting, and/or reducing a cancer or metastasis in a subject comprising administering to the subject any of the PD-L1 binding molecules (including, but not limited to neutralizing PD-L1 binding molecules such as, for example, neutralizing anti-PD-L1 antibodies) disclosed herein. For example, in one aspect, disclosed herein are methods of treating, preventing, inhibiting, and/or reducing a cancer or metastasis in a subject comprising administering to the subject an isolated PD-L1 binding molecule (including, but not limited to neutralizing PD-L1 binding molecules such as, for example, neutralizing anti-PD-L1 antibodies) comprising a heavy chain variable domain comprising one or more Complementary Determining Regions (CDR)s as set forth in Table 1. Thus, for example, the methods of treating, preventing, inhibiting, and/or reducing a cancer or metastasis in a subject can comprise administering to the subject a PD-L1 binding molecule (including, but not limited to neutralizing PD-L1 binding molecules such as, for example, neutralizing anti-PD-L1 antibodies) comprising one or more heavy chain variable domain CDRs can comprise SEQ ID NO: 3, SEQ ID NO: 4, SEQ ID NO: 5, SEQ ID NO: 6, SEQ ID NO: 7, SEQ ID NO: 8, SEQ ID NO: 9, and/or SEQ ID NO: 10. In one aspect, it is understood and herein contemplated that the PD-L1 binding molecules (including, but not limited to neutralizing PD-L1 binding molecules such as, for example, neutralizing anti-PD-L1 antibodies) used in the disclosed methods of treating, preventing, inhibiting, and/or reducing a cancer or metastasis in a subject can comprise heavy chain variable domain CDR comprising any combination of 2 or 3 heavy chain variable domain CDRs from the list of CDRs in Table 1. Thus, for example, the methods of treating, preventing, inhibiting, and/or reducing a cancer or metastasis in a subject can comprising administering PD-L1 binding molecules (including, but not limited to neutralizing PD-L1 binding molecules such as, for example, neutralizing anti-PD-L1 antibodies) comprising the heavy chain variable domain CDRs as set forth in SEQ ID NO: 3 and SEQ ID NO: 5; SEQ ID NO: 3 and SEQ ID NO: 6; SEQ ID NO: 3 and SEQ ID NO: 7; SEQ ID NO: 3 and SEQ ID NO: 8; SEQ ID NO: 3 and SEQ ID NO: 9; SEQ ID NO: 3 and SEQ ID NO: 10; SEQ ID NO: 4 and SEQ ID NO: 5; SEQ ID NO: 4 and SEQ ID NO: 6; SEQ ID NO: 4 and SEQ ID NO: 7; SEQ ID NO: 4 and SEQ ID NO: 8; SEQ ID NO: 4 and SEQ ID NO: 9; SEQ ID NO: 4 and SEQ ID NO: 10; SEQ ID NO: 3, SEQ ID NO: 5, and SEQ ID NO: 9; SEQ ID NO: 3, SEQ ID NO: 6, and SEQ ID NO: 9; SEQ ID NO: 3, SEQ ID NO: 7, and SEQ ID NO: 9; SEQ ID NO: 3, SEQ ID NO: 8, and SEQ ID NO: 9; SEQ ID NO: 3, SEQ ID NO: 5, and SEQ ID NO: 10; SEQ ID NO: 3, SEQ ID NO: 6, and SEQ ID NO: 10; SEQ ID NO: 3, SEQ ID NO: 7, and SEQ ID NO: 10; SEQ ID NO: 3, SEQ ID NO: 8, and SEQ ID NO: 10; SEQ ID NO: 4, SEQ ID NO: 5, and SEQ ID NO: 9; SEQ ID NO: 4, SEQ ID NO: 6, and SEQ ID NO: 9; SEQ ID NO: 4, SEQ ID NO: 7, and SEQ ID NO: 9; SEQ ID NO: 4, SEQ ID NO: 8, and SEQ ID NO: 9; SEQ ID NO: 4, SEQ ID NO: 5, and SEQ ID NO: 10; SEQ ID NO: 4, SEQ ID NO: 6, and SEQ ID NO: 10; SEQ ID NO: 4, SEQ ID NO: 7, and SEQ ID NO: 10; or SEQ ID NO: 4, SEQ ID NO: 8, and SEQ ID NO: 10. In one aspect, the methods of treating, preventing, inhibiting, and/or reducing a cancer or metastasis in a subject can comprise administering to the subject a PD-L1 binding molecule (including, but not limited to neutralizing PD-L1 binding molecules such as, for example, neutralizing anti-PD-L1 antibodies) comprising the variable heavy domain as set forth in SEQ ID NO: 1.

123. In one aspect, disclosed herein are methods of treating, preventing, inhibiting, and/or reducing a cancer or metastasis in a subject comprising administering to the subject an isolated PD-L1 binding molecule (including, but not limited to neutralizing PD-L1 binding molecules such as, for example, neutralizing anti-PD-L1 antibodies) comprising a light chain variable domain comprising one or more Complementary Determining Regions (CDR)s as set forth in Table 1. Thus, for example, the methods of treating, preventing, inhibiting, and/or reducing a cancer or metastasis in a subject can comprise administering to the subject a PD-L1 binding molecule (including, but not limited to neutralizing PD-L1 binding molecules such as, for example, neutralizing anti-PD-L1 antibodies) comprising one or more light chain variable domain CDRs can comprise SEQ ID NO: 11, SEQ ID NO: 12, SEQ ID NO: 13, SEQ ID NO: 14, and/or SEQ ID NO: 15. In one aspect, it is understood and herein contemplated that the PD-L1 binding molecules (including, but not limited to neutralizing PD-L1 binding molecules such as, for example, neutralizing anti-PD-L1 antibodies) used in the disclosed methods of treating, preventing, inhibiting, and/or reducing a cancer or metastasis in a subject can comprise light chain variable domain CDR comprising any combination of 2 or 3 light chain variable domain CDRs from the list of CDRs in Table 1. Thus, for example, the methods of treating, preventing, inhibiting, and/or reducing a cancer or metastasis in a subject can comprising administering PD-L1 binding molecules (including, but not limited to neutralizing PD-L1 binding molecules such as, for example, neutralizing anti-PD-L1 antibodies) comprising the light chain variable domain CDRs as set forth in SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15. In one aspect, the methods of treating, preventing, inhibiting, and/or reducing a cancer or metastasis in a subject can comprise administering to the subject a PD-L1 binding molecule (including, but not limited to neutralizing PD-L1 binding molecules such as, for example, neutralizing anti-PD-L1 antibodies) comprising the variable light chain domain as set forth in SEQ ID NO: 2. In one aspect, the PD-L1 binding molecule comprises the variable heavy chain domain as set forth in SEQ ID NO: 1 and the variable light chain domain as set forth in SEQ ID NO: 2.

124. As used herein the terms “treatment,” “treat,” or “treating” refers to a method of reducing one or more of the effects of a disease or condition (such as, for example an inflammatory condition or a cancer) in the subject. Thus in the disclosed method, treatment can refer to a 10%, 20%, 30%, 40%, 50%, 60%, 70%, 80%, 90%, or 100% reduction in the severity of an established infection or a symptom of the infection. For example, a method for treating an inflammatory condition or cancer is considered to be a treatment if there is a 10% reduction in one or more symptoms of the condition or cancer in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the condition or disease or symptoms of the condition or disease. It is understood and herein contemplated that treatments as discussed herein can be prophylactic or therapeutic. Accordingly, in one aspect are methods of treating or reducing the severity of an inflammatory disease or condition in a subject comprising administering to the subject an PD-L1 binding molecule. Also disclosed are methods of preventing or reducing the onset of an inflammatory disease or condition in a subject comprising administering to the subject an PD-L1 binding molecule.

125. As used herein, the terms prevent, preventing, and prevention of an infection, refers to an action, for example, administration of a therapeutic agent (e.g., a composition disclosed herein), that occurs before or at about the same time a subject begins to show one or more symptoms of the infection, which inhibits or delays onset or exacerbation or delays recurrence of one or more symptoms of the infection. As used herein, references to decreasing, reducing, or inhibiting include a change of 10%, 20%, 30%, 40%, 50%, 60%, 70%, 80%, 90% or greater relative to a control level. For example, the disclosed methods are considered to be a prevention if there is about a 10% reduction in onset, exacerbation or recurrence of inflammatory condition or a disease, or symptoms of an inflammatory condition or a disease in a subject when compared to control subjects that did not receive an PD-L1 binding molecule for decreasing the inflammatory condition or disease.

126. It is understood and herein contemplated that the disclosed methods of treating, preventing, inhibiting, or reducing a cancer or metastasis in a subject comprising administering any of the PD-L1 binding molecules disclosed herein (including, but not limited to neutralizing PD-L1 binding molecules such as, for example, neutralizing anti-PD-L1 antibodies) can further comprise the administration of any anti-cancer agent that would further aid in the reduction, inhibition, treatment, and/or elimination of the cancer or metastasis (such as, for example, gemcitabine). Anti-cancer agents that can be used in the disclosed bioresponsive hydrogels or as an additional therapeutic agent in addition to the disclosed pharmaceutical compositions, and/or bioresponsive hydrogel matrixes for the methods of reducing, inhibiting, treating, and/or eliminating a cancer or metastasis in a subject disclosed herein can comprise any anti-cancer agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

### C. EXAMPLES

127. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

128. Transgenic mice overexpressing mouse Ig-Alpha, mouse Ig-Beta and human interleukin 6 were injected intraperitoneally with recombinant human PD-L1 (R&D Systems) at 2 week intervals. After a significant immune response was mounted as measured by serum ELISA, the lymph nodes, spleens and bone marrow cells were harvested, B cells surface-expressing IgM isotype antibodies were subtracted with magnetic beads, and the remaining cells were sorted for their ability to bind PD-L1, using a MoFlo Fluorescence—Activated Cell Sorter.

129. Cells positive for PD-L1 binding were sorted into 96-well plates, subjected to single cell RT-PCR to amplify variable regions, and the variable regions were cloned into expression vectors containing either a heavy chain human IgG1 or IgG4 constant region or light chain human IgK constant region. The resulting heavy and light chain clone pairs were transfected into CHO cells, and the resulting antibody protein was purified with protein A resin.

130. The ability of the antibodies to bind native cell-surface PD-L1 was measured using flow cytometry. In brief, purified antibodies were mixed with cells individually, and a fluorescently-labeled secondary antibody was added to the cell-antibody mixture. When the cells were passed through the flow cytometer, the antibodies specific for binding to PD-L1 significantly increased the fluorescence of the cells.

131. The ability of the antibodies to disrupt the interaction of a cell displaying on its surface membrane-bound PD-L1 to another cell displaying on its surface membrane-bound PD-1 was evaluated in an in vitro cell reporter assay, and IC50 concentrations were calculated (Table 2).

TABLE 2
the identification number and IC50 of activity of each antibody
in a two-cell blocking assay of the PD-L1/PD-1 interaction

| Name | IC50 |
|---|---|
| ABM101 | 0.05 |
| ABM102 | 0.32 |
| ABM103 | 0.08 |
| ABM118 | 0.08 |
| ABM139 | 0.08 |

*IC50 shown in micrograms per milliliter

132. ABM101 had the best potency in the PD-1/PD-L1 reporter assay and was chosen for humanization. CDRs were grafted onto a variety of human frameworks, and ABM101.11 was the most potent humanized antibody with the best expression levels (Table 3).

TABLE 3
the identification number, human isotype, IC50, dissociation
constant against human PD-L1, and dissociation constant
against cynomolgus monkey of humanized variants of a
selected mouse antibody designated ABM101.

| | | | KD by SPR | KD by SPR |
|---|---|---|---|---|
| hABM# | Isotype | IC50 | (HUMAN) | (CYNO) |
| ABM101-G4 | IgG4 | 0.05 | | |
| ABM101-G1 | IgG1 | 0.19 | | |
| hABM101.1.1 | IgG4 | 0.52 | | |
| hABM101.1.2 | IgG4 | 0.62 | | |
| hABM101.1.3 | IgG4 | 0.18 | | |
| hABM101.2.3 | IgG4 | 0.28 | | |
| hABM101.1.4 | IgG4 | 0.05 | | |
| hABM101.2.4 | IgG4 | 0.26 | | |
| hABM101.3.1-G1 | IgG1 | 0.07 | | |
| hABM101.3.1-G4 | IgG4 | 0.07 | | |
| hABM101.4.1 | IgG4 | 0.05 | | |
| hABM101.3.5 | IgG4 | 0.21 | | |
| hABM101.7 | IgG4 | 0.02 | 31 pM | 39 pM |
| hABM101.7 | IgG1 | 0.046 | | |
| hABM101.8 | IgG4 | 0.028 | 109 pM | 143 pM |
| hABM101.8 | IgG1 | 0.058 | | |
| hABM101.9 | IgG4 | 0.019 | 152 pM | 171 pM |
| hABM101.9 | IgG1 | 0.022 | | |
| hABM101.11 | IgG4 | 0.018 | 349 pM | |
| hABM101.11 | IgG1 | 0.023 | 374 pM | 579 pM |

### Sequences

SEQ ID NO: 1: ABM101.11 Heavy Chain
QVQLVQSGpEVKKPGASVKVSCKaSGYTFTENSMHWVrQSHGKsLEWmGG
INPNNGGTSYNQKFKGkvTmTTDKSTSTAYMELRSLTSDDTAVYYCARPY
YYGYREDYFDYWGQGTTLTVSS
SEQ ID NO: 2: ABM101.11 Light Chain
EIVMTQSPGTLSLSPGERATLSCRASSSVSYMYWYQQKPGQAPRPLIYLT
SNLASGIPDRFSGSGSGTDYTLTISRLEPEDFAVYYCQQWSSYPPTFGQG
TKVEIKR

1. An isolated PD-L1 binding molecule comprising a heavy chain variable domain comprising at least one CDR as set forth in SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

2. The isolated PD-L1 binding molecule of claim 1, wherein the binding molecule comprising a heavy chain variable domain comprises the CDRs as set forth in SEQ ID NO: 3 and SEQ ID NO: 5; SEQ ID NO: 3 and SEQ ID NO: 6; SEQ ID NO: 3 and SEQ ID NO: 7; SEQ ID NO: 3 and SEQ ID NO: 8; SEQ ID NO: 3 and SEQ ID NO: 9; SEQ ID NO: 3 and SEQ ID NO: 10; SEQ ID NO: 4 and SEQ ID NO: 5; SEQ ID NO: 4 and SEQ ID NO: 6; SEQ ID NO: 4 and SEQ ID NO: 7; SEQ ID NO: 4 and SEQ ID NO: 8; SEQ ID NO: 4 and SEQ ID NO: 9; SEQ ID NO: 4 and SEQ ID NO: 10; SEQ ID NO: 3, SEQ ID NO: 5, and SEQ ID NO: 9; SEQ ID NO: 3, SEQ ID NO: 6, and SEQ ID NO: 9; SEQ ID NO: 3, SEQ ID NO: 7, and SEQ ID NO: 9; SEQ ID NO: 3, SEQ ID NO: 8, and SEQ ID NO: 9; SEQ ID NO: 3, SEQ ID NO: 5, and SEQ ID NO: 10; SEQ ID NO: 3, SEQ ID NO: 6, and SEQ ID NO: 10; SEQ ID NO: 3, SEQ ID NO: 7, and SEQ ID NO: 10; SEQ ID NO: 3, SEQ ID NO: 8, and SEQ ID NO: 10; SEQ ID NO: 4, SEQ ID NO: 5, and SEQ ID NO: 9; SEQ ID NO: 4, SEQ ID NO: 6, and SEQ ID NO: 9; SEQ ID NO: 4, SEQ ID NO: 7, and SEQ ID NO: 9; SEQ ID NO: 4, SEQ ID NO: 8, and SEQ ID NO: 9; SEQ ID NO: 4, SEQ ID NO: 5, and SEQ ID NO: 10; SEQ ID NO: 4, SEQ ID NO: 6, and SEQ ID NO: 10; SEQ ID NO: 4, SEQ ID NO: 7, and SEQ ID NO: 10; or SEQ ID NO: 4, SEQ ID NO: 8, and SEQ ID NO: 10.

3. The isolated PD-L1 binding molecule of claim 2 comprising the variable heavy domain as set forth in SEQ ID NO: 1.

4. The isolated PD-L1 binding molecule of claim 1, further comprising a light chain variable domain comprising at least one CDR as set forth in SEQ ID NO: 11, SEQ ID NO: 12, SEQ ID NO: 13, SEQ ID NO: 14, or SEQ ID NO: 15.

5. The isolated PD-L1 binding molecule of claim 4, wherein the binding molecule comprising a light chain variable domain comprises the CDRs as set forth in SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15.

6. The isolated PD-L1 binding molecule of claim 5 comprising the variable light domain as set forth in SEQ ID NO: 2.

7. The isolated PD-L1 binding molecule of claim 6 wherein the binding molecules comprises a variable heavy domain as set forth in SEQ ID NO: 1.

8. An isolated PD-L1 binding molecule comprising a light chain variable domain comprising at least one CDR as set forth in SEQ ID NO: 11, SEQ ID NO: 12, SEQ ID NO: 13, SEQ ID NO: 14, or SEQ ID NO: 15.

9. The isolated PD-L1 binding molecule of claim 8, wherein the binding molecule comprising a heavy chain variable domain comprises the CDRs as set forth in SEQ ID NO: 11 and SEQ ID NO: 13; SEQ ID NO: 11 and SEQ ID NO: 14; SEQ ID NO: 11 and SEQ ID NO: 15; SEQ ID NO: 12 and SEQ ID NO: 13; SEQ ID NO: 12 and SEQ ID NO: 14; SEQ ID NO: 12 and SEQ ID NO: 15; SEQ ID NO: 11, SEQ ID NO: 13, and SEQ ID NO: 14; SEQ ID NO: 11, SEQ ID NO: 13, and SEQ ID NO: 15; SEQ ID NO: 12, SEQ ID NO: 13, and SEQ ID NO: 14; or SEQ ID NO: 12, SEQ ID NO: 13, and SEQ ID NO: 15.

10. The isolated PD-L1 binding molecule of claim 1 wherein the binding molecule is an antibody.

11. The isolated PD-L1 binding molecule of claim 1, wherein the binding molecule is an immunotoxin.

12. An antibody containing or comprising a heavy chain variable region as set forth in SEQ ID NO: 1 or a light chain variable region as set forth in SEQ ID NO: 2.

13. (canceled)

14. The antibody of claim 12, containing or comprising the heavy chain variable region as set forth in SEQ ID NO: 1 and further comprising the light chain variable region as set forth in SEQ ID NO: 2.

15. The isolated PD-L1 binding molecule of claim 10, wherein the isolated binding molecule is a neutralizing antibody.

16. A method of treating or preventing cancer or cancer-related diseases of cell proliferation by administering an amount of any of the PD-L1 binding molecules of claim 1.

17. The isolated PD-L1 binding antibody of claim 12, wherein the antibody is a neutralizing antibody.

18. The isolated PD-L1 binding molecule of claim 8, wherein the binding molecule is an antibody or immunotoxin.

19. The isolated PD-L1 binding antibody of claim 18, wherein the antibody is a neutralizing antibody.

20. A method of treating or preventing cancer or cancer-related diseases of cell proliferation by administering an amount of the antibody of claim 12.

21. A method of treating or preventing cancer or cancer-related diseases of cell proliferation by administering an amount of any of the PD-L1 binding molecules of claim 8.
